(19) United States
(12) Patent Application Publication
(10) Pub. No.: US 2004/0064112 A1
(43) Pub. Date: Apr. 1, 2004

(54) Disposable personal urinal with heat-sealed anti-backflow tri-valve and folded-in edge mouth

(76) Inventor: Robert Sun, Fremont, CA (US)

Correspondence Address: Robert Sun, 2900 Sanderling Dr., Fremont, CA, 94555, US

(21) Appl. No.: 10/255,192

(22) Filed: Sep. 27, 2002

(51) Int. Cl. A61F 5/44

(52) U.S. Cl. 604/346; 604/347

(57) ABSTRACT

A collection device for human bodily fluids includes a conventional bag constructed of a soft plastic, waterproof material. The bag consists of a hard plastic supporting mouth, proper volume of hydrophilic material such as polymer and a heat-sealed anti-backflow tri-valve in the interior of a front trunk portion of the bag. The supporting mouth is located as to cover the genitourinary area or mouth of an individual in use for urinal or vomiting purposes. This hydrophilic material allows quick absorption of human fluids and turns it into a gelled material. The gelled material is then trapped inside the heat-sealed tri-valve bag to prevent possible spillage and backflow. The versatility of the heat-sealed funnel design bag helps the direct engagement with the hydrophilic material to speed up absorption time with the human fluid and preventing spillage or backflow. It also simplifies the manufacturing process and reduces overall production costs.

### BACKGROUND OF THE INVENTION

1. Field of the Invention

This invention relates to a method of producing a low-cost and versatile human bodily fluid collection device serving as a car sick storage bag or a portable urinal, it is excellent in its ease of use for men and women, and more particularly to a portable urinal provided with a folded-in edge mouth element so located as to cover the mouth or genitourinary area of the user and for the prevention of accident spillage or backflow. This device encloses a proper volume of hydrophilic powder or granular material for absorbing the fluid. This hydrophilic powder or granule serves the purpose of absorbing any incoming fluid to the device and turns them into an expanding pile of soft gel. A tri-valve heated seal urine bag constructed of a waterproof material serves to prevent the hydrophilic material from leaking out of the collection device.

2. Description of the Prior Art

The improvement of this invention is directed to the conventional urine bag as disclosed in U.S. Pat. No. 5,007,116. The conventional urine bag has a urine impermeable bag body made of a soft plastic, a support made of a hard plastic, and an absorbing pouch made of an absorbent material which can absorb urine. The bag body has an accommodating chamber and an open upper end hot sealed on the support. The support has a central hole formed therethrough and is communicated with the accommodating chamber. The absorbing body is received in the accommodating chamber of the bag body.

The urine bag consists of the following drawbacks:

(a) Because the urine bag contains a pouch which is constructed of a water-permeable material that is filled with a water-absorbing agent which is swollen with urine to form a gel when it is brought into contact with said urine to absorb the same, said pouch being broken into fragments when said water-absorbing agent is swollen with said urine, this process will slow down the overall absorbent time in comparison to the direct engagement with the water absorbent agent, said hydrophilic material. Furthermore, it is cost effective to omit the pouch inside the urine bag, which would also substantially increase the efficiency of the manufacturing process and cut down the overall production cost.

(b) This design poses another problem. Once the pouch breaks apart, the gel can move freely around inside the bag. Although the bag can be sealed from the upper portion, any puncture at any given location can still allow the gel to leak out of the bag. Any person who seals this bag manually may risk the possibility of coming into contact with the bodily fluid waste.

Yet another problem is that there is a possibility of spilling the gel during use if the urinal is accidentally dropped or squeezed too hard from the outside.

Another form of the bodily fluid collection device, known as the “Little John”, is a widely used urinal in hospital for many years. This urinal intended to be portable, reusable, convenient and spill-proof, is constructed of a durable hard plastic, with a handle for carrying, a screw-on cap for covering and an optional “Lady J Adapter” for female use. Although the cap can be screwed on to prevent spillage, the disadvantage of this design is that during its use, dropping this urinal will spill the urine before this cap can even be used. Also, when this Lady J Adapter is used, the cap cannot be screwed on, which is another possibility of allowing urine to spill during use or transport.

None of the above devices provides a complete assurance of preventing backflow, leakage and spill problems nor can it be cheaply manufactured and assembled. It is, therefore, an object of invention to devise a simple yet useful method of collecting and absorbing the human bodily fluid that provides a folded-in edge mouth element and effective heat-sealed tri-valve to prevent leakage and backflow.

Yet another object of the invention is to provide the largest capacity of absorption power while maintaining the advantage of being small, compact and lightweight.

A further object of the invention is to manufacture this method inexpensively and to increase its ease of transport, handling and storage with an assurance of being leakage proof and backflow proof.

### SUMMARY OF THE INVENTION

This invention has been devised to offer a simple yet useful method of collecting and absorbing human bodily fluids that prevents leakage and backflow by utilizing a folded-in edge mouth element so located as to cover the mouth or genitourinary area of the user and for the prevention of accidental spillage or backflow. A heat-sealed tri-valve provided in the interior of a front trunk portion of the bag prevents the backflow of the inner hydrophilic material that allows quick absorption of such fluids and turns into gelled material. The gelled material is then trapped inside the heat-sealed tri-valve bag to prevent possible spills and backflow. The hydrophilic material with a proper volume allows expansion up to the predetermined amount of gel to fit in different types of human bodily fluid collection and reduce possible leakage and backflow.

The present invention provides the following portable urinal:

A portable urinal comprising of a urine storage bag constructed of a soft plastic, waterproof material; and a folded-in edge mouth element so located as to cover the mouth or genitourinary area of the user, and a heat-sealed tri-valve provided in the interior of a front trunk portion of the bag to prevent the backflow of the inner hydrophilic material and is associated with a snap-fit seal from the upper portion of the device. It is assured to hold up all the expanded gel without breaking. Therefore, the problems of the fluid leaking, the gel clogging the collection inlet, the gel leaking out a punctured bag, or the gel flowing backwards and spilling out from the device inlet are controlled, and the possibilities of any spillage or leakage are substantially minimized.

### BRIEF DESCRIPTION OF THE DRAWING

The accompanying drawings show other features and advantages of this human bodily fluids collection device:

FIG. 1 is a schematic view illustrating the use of the invented (1) portable urinal device, that has a (2) supporting mouth made of hard plastic with a (3) folded-in edge mouth element for the prevention of any potential liquid backflow; a (4) heat-sealed anti-backflow tri-valve on the (5) bag body, the (5) bag body is filled with (6) liquid absorbent polymer according to this invention.

### DESCRIPTION OF THE PREFERRED EMBODIMENTS

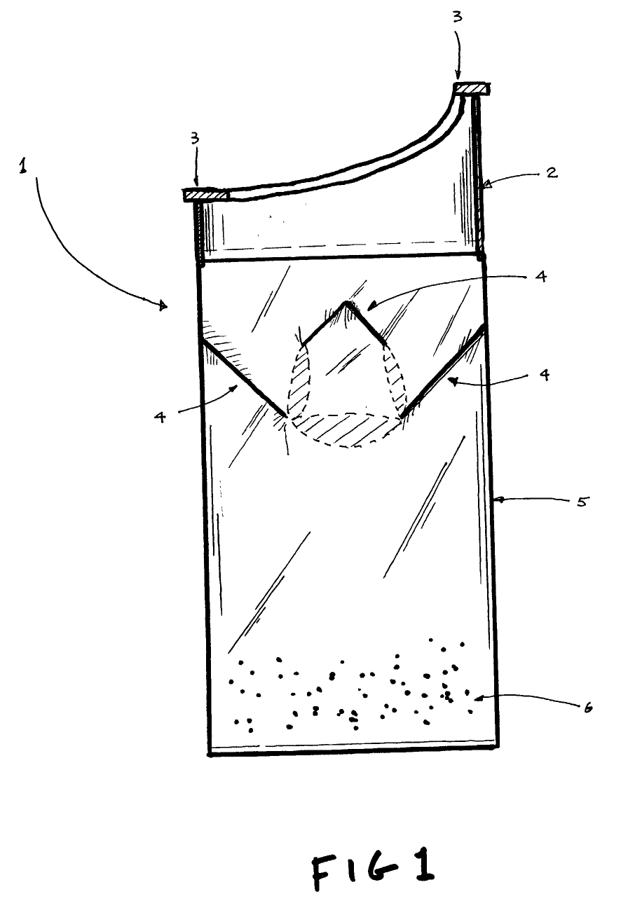

FIG. 1 illustrates a (1) human bodily fluid collection device with a (2) large adapter inlet consists of a hard plastic contour shape and a (3) folded-in edge mouth that can be used by male or female. The (3) folded-in edge mouth element so located as to cover the mouth or genitourinary area of the user and a (5) urine bag with a (4) heat-sealed anti-backflow tri-valve on its said (5) bag body as in accordance with the principles of this invention.

The urinal is constructed of a said (2) large adapter inlet, a (5) liquid collection bag with a said (4) heat-sealed anti-backflow tri-valve. The (2) large adapter inlet consists of a hard plastic contour shape with a (3) folded-in edge mouth that can be used by male or female. The effectiveness of the present invention can be clearly seen when the urine flow into the (5) collection bag, the (3) folded-in edge mouth of this device will prevent the urine and gel from flowing backward. Also even if the (5) bag is punctured, the (4) heat-sealed tri-valve on the said (5) bag body will still hold back the (6) powder absorbent agent or gel material inside the (5) collection bag body acting as a double protection, if required, the collection bag can be constructed with an optional snap-fit fastener to prevent leakage of excess liquid inside the bag.

I claim:

1. A human bodily fluid collecting device for the purpose of collecting and absorbing said bodily fluid, comprising a container which has one opening inlet through which human bodily fluid enters said container, and which at least partially encloses said heat-sealed tri-valve to prevent direct contact with human body.

2. The human bodily collecting device as claimed in claim 1 in which said container mean is selected from the group consisting of a urine collection reservoir, a portable urinal, a bedpan, a urostomy bag, an ileostomy bag, or an ostomy bag.

3. The human bodily fluid collection device as claimed in claim 2 in which said portable urinal is made of a portable urinal material selected from the group consisting of hard plastic, soft plastic, impermeable paper, and combinations of portable urinal materials.

4. The human bodily fluid collection device as claimed in claim 1 which has one large adapter inlet say adapter inlet is made of hard plastic in contour shape with a folded-in mouth that can be used by male or female to prevent the urine and gel backflow when using the device.

5. A method of using a heat-sealed tri-valve on the collection device wherein when a bodily fluid enters said bag body through the heat-sealed tri-valve and engage with said liquid permeable material said hydrophilic material, causing said hydrophilic material expands and turns into a soft gel; and encapsulating said soft gel inside said human bodily collection device with heat-sealed tri-valve to prevent said soft gel from escaping said urine bag without the leakage.

6. The method as claimed in claim 5 wherein said hydrophilic material is a super absorbent polymer and said step of causing said fluid to form said soft gel comprises the step of contacting said fluid with said super absorbent polymer.
